(19) United States
(12) Patent Application Publication
(10) Pub. No.: US 2010/0240112 A1
(43) Pub. Date: Sep. 23, 2010

(54) Process for Preparing a Sugar Product

(76) Inventors: Juha Anttila, Oulu (FI); Juha Tanskanen, Oulu (FI); Pasi Rousu, Oulu (FI); Päivi Rousu, Kotka (FI); Keijo Hytönen, Oulu (FI)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/741,693

(22) PCT Filed: Nov. 7, 2008
(86) PCT No.: PCT/FI08/50639

§ 371 Date: May 6, 2010

(30) Foreign Application Priority Data
Nov. 9, 2007 (FI) 20075795

(51) Int. Cl. C12P 7/14; C12P 7/24

(52) U.S. Cl. 435/147; 435/162

(57) ABSTRACT

The invention relates to a method for producing sugars, such as glucose, by fractionating lignocellulose-containing biomass. The sugar product thus obtained is useful for the manufacture of bioethanol and other chemicals.

### BACKGROUND OF THE INVENTION

The invention relates to a method for producing sugars, such as glucose, from lignocellulose-containing biomass. The sugar product thus obtained is useful for the preparation of bioethanol and other chemicals.

At present, there are several reasons for the need to manufacture ethanol from a raw material containing lignocellulose. Firstly, there is demand for biofuel for traffic, and, secondly, there is need for second-generation solutions, i.e. techniques enabling the utilization of the entire biomass, i.e. lignocellulose, of a plant, not only a given part of the plant, such as the grain, the sugars or the oil, for example. In addition, ethanol produced from carbohydrates is at present the most generally used biofuel in traffic, and also one of the most potential future alternatives.

In addition to ethanol, other such chemicals are attempted to be produced from lignocellulose that are at present produced from non-renewable natural resources. Sugars hydrolyzed from lignocellulose are potential starting materials for such production.

Hamelinck et al./1/ extensively compared the second-generation ethanol production methods presented and potential future methods. These methods face a plurality of challenges.

In order for known second-generation ethanol production methods to be profitable, all carbohydrates (cellulose and hemicelluloses) of lignocellulose are attempted to be hydrolyzed into sugars and further fermented at a high yield into ethanol. The hydrolysis of cellulose and hemicelluloses into sugars requires different pre-treatment and hydrolysis conditions. The fermentation of glucose generated from cellulose into ethanol is known art, but the fermentation of pentoses typically generated from hemicelluloses is not yet mature art. In addition, a significant part of the sugars in hemicelluloses is typically lost in pre-treatment, which impairs the yield of ethanol.

In known methods, pre-treatment does not generally remove lignin, lignin being present in the hydrolysis of cellulose, which complicates the operation of enzymatic hydrolysis /2/.

In many methods, sulphuric acid, either as diluted /2/ or concentrated /1, 3/, is used in pre-treatment and hydrolysis. If sulphuric acid is used diluted, it is not attempted to be recovered, but is neutralized, whereby gypsum waste /1/ is generated. If concentrated sulphuric acid is used, it has to be recovered and recycled, but the recovery requires a complex chromatographic separation /1, 3/.

In some pre-treatment methods, e.g. steam explosion, a high temperature (about 200° C.) and high pressures bring about special challenges /1/.

Typically, the hydrolysis of cellulose is proposed to be implemented enzymatically. In addition to the yield of ethanol, the profitability of the methods is vitally affected by the amount of enzyme used and the investment cost of the hydrolysis step, which, in turn, depends on the residence time required for the hydrolysis /1/.

In known methods, also the treatment of the unhydrolyzed fraction (mainly lignin) requires high investments and consumes energy /1/.

The use of organic acids, such as formic acid and acetic acid, for example, for fractionating lignocellulose for the manufacture of pulp and paper is known /4 to 9/. In this case, the cellulose of the raw material and part of the hemicelluloses remain in the pulp fraction, whereas lignin and the rest of the hemicelluloses are dissolved in the cooking liquor. To facilitate bleaching, the attempt is to remove lignin from the pulp as carefully as possible without the lignin starting to condensate. Hemicelluloses remain in the pulp and they improve the paper-technical properties of the pulp /12/. A corresponding use of ethanol as a pulp production chemical is known /10/, and ethanol may be used as a pre-treatment chemical when sugars and, further, ethanol, are produced from lignocellulose /11/.

### BRIEF DESCRIPTION OF THE INVENTION

It has now been unexpectedly found that a water-soluble sugar product may be advantageously produced from lignocellulose-containing biomass by a method comprising first selectively fractioning the biomass with a reagent containing organic acids. This gives a solid carbohydrate product having improved hydrolyzability, which is then hydrolyzed enzymatically into water-soluble oligosaccharides and monosaccharides, such as glucose, for example. The characteristics of the solid carbohydrate product obtained as an intermediate differ from those of pulp manufactured for paper making, but it is excellently suitable as a raw material for glucose and, further, ethanol or other chemicals. The hydrolyzability of the carbohydrate fraction can be further improved by subjecting the fraction to further treatments with a reagent containing organic acids in the steps following the fractionation.

When biomass is fractioned in the manner described in the present invention, the lignin and hemicelluloses contained by the biomass are mainly dissolved into the acid mixture used in the fractionation. Organic cooking acids may be recovered from this mixture simply by known methods, and furfural, acetic acid, formic acid, chemicals and biofuel may be produced from the lignin and the hemicelluloses. The combination of a simple recovery of organic acids and the production of chemicals with the production of an excellent carbohydrate fraction results in extremely productive biorefining.

### DETAILED DESCRIPTION OF THE INVENTION

The invention relates to a method for producing sugars, such as glucose, from lignocellulose-containing biomass. The method is characterized by (A) treating the biomass with a reagent containing one or more organic acids, yielding a solid carbohydrate fraction having improved hydrolyzability, and a fraction/fractions containing organic matter dissolved from the biomass and used organic acids, and (B) enzymatically hydrolyzing at least part of the solid carbohydrate fraction obtained into water-soluble monosaccharides and oligosaccharides, yielding a sugar product. After the treatment of step (A), the method comprises conventional separation steps, wherein the solid carbohydrate product is separated from the other fractions obtained.

The biomass used as the starting material of the method may be any lignocellulose-containing plant material. It may be wood material, such as conifer or deciduous wood. It may also be non-wood material based on grass-stemmed plants, bast fibres, leaf fibres or fruit fibres. Examples of suitable materials based on grass-stemmed plants include straw, e.g. cereal straw (wheat, rye, oat, barley, rice), reeds, e.g. reed canary grass, common reed, papyrus, sugar cane, i.e. bagasse and bamboo, and grasses, e.g. esparto, sabai and lemon grass. Examples of bast fibres include flax, such as stems of common flax and stems of oil flax, hemp, East Indian hemp, kenaf, jute, ramie, paper mulberry, gampi fibre and mitsumata fibre. Examples of leaf fibres include abaca and sisal, among others. Examples of fruit fibres include cotton reed hairs and cotton linter fibres, capoc and coir fibre.

Out of grass-stemmed plants growing in Finland and useful in the present invention, common reed, reed canary grass, timothy, cocksfoot grass, yellow sweet clover, smooth brome, red fescue, white sweet clover, red clover, goat's rue and alfalfa, may be mentioned.

Biomass based on grass-stemmed plants, such as cereal straw, is used particularly advantageously. In an embodiment, biomass based on annual grass-stemmed plants is used. In another embodiment, biomass based on perennial non-wood plants is used. In accordance with the invention, agricultural waste material may also be used, including, inter alia, the above-mentioned cereal straw.

In the treatment of step (A), a reagent based on organic acids, such as formic, acetic and/or propionic acid is used. A reagent containing formic acid and/or acetic acid is preferably used. A reagent containing formic acid is particularly preferably used. The amount of formic acid and acetic acid may vary within the range 0 to 95%. In addition to formic acid and acetic acid, the reagent typically contains water, typically within the range 5 to 50%. In a preferred embodiment, the treatment reagent contains less than 60% acetic acid, the rest being formic acid and water. In another preferred embodiment, the treatment reagent contains less than 60% acetic acid and at least 20%, typically 40 to 95% formic acid. In still another embodiment, the treatment reagent contains less than 40% acetic acid and at least 40% formic acid.

If desired, other acids, such as sulphuric acid or hydrochloric acid or organic peroxide acids, for example, may also be used.

The treatment temperature of step (A) is typically within the range 60 to 220° C. In a preferred embodiment, the temperature is within the range 100 to 180° C., 130 to 170° C., for example. The treatment time may be within the range 5 minutes to 10 h, typically 15 minutes to 4 h.

In step (A) of the method of the invention, a solid carbohydrate fraction having improved hydrolyzability is obtained and it was found to be hydrolyzed into water-soluble sugars faster and with a smaller amount of enzyme than conventional pulp produced for paper making.

The carbohydrates of the solid carbohydrate fraction obtained in step (A) of the method of the invention contain mainly (typically at least 80%) polysaccharides composed of units formed by glucose and other hexoses, and, in addition, preferably less than 10%, e.g. less than 5% polysaccharides composed of pentose units. These pentoses are typically mainly xylose and arabinose. The carbohydrate fraction contains both fibrous and non-fibrous matter.

The lignin content of the solid carbohydrate fraction obtained in step (A) of the method of the invention is low, i.e. the kappa number is typically less than 50.

In the fractionation step (A) of the method of the invention, a fraction or fractions are also obtained that contain organic matter dissolved from the biomass and organic acids used in the treatment. The organic matter dissolved from the biomass typically contains lignin and sugars of hemicelluloses, such as hexoses and pentoses. The separated, dissolved organic matter is useful, inter alia, as biofuel or raw material for gasification, for example.

The solid carbohydrate fraction obtained is separated from the other fractions obtained, such as from the dissolved organic matter, by known methods, filtering, washing or pressing, for example. In these methods, organic acids circulating in the process or mixtures thereof may be used as auxiliary agents. The organic acids remaining in the solid carbohydrate fraction may be separated by the same known methods, whereby water may be used as the auxiliary agent in the separation.

When the different fractions are separated by washing, the washing may typically be carried out in two steps for instance by performing the washing first with a concentrated acid and then with water. The concentrated acid used in the first washing step may be the same as the acid mixture used for the fractionation.

The solid carbohydrate fraction thus obtained in step (B) of the method of the invention or a part of this fraction is enzymatically hydrolyzed into water-soluble monosaccharides and oligosaccharides, and, if desired, further into monosaccharides, whereby the sugar product according to the invention is obtained. In connection with the present invention, water-soluble oligosaccharides refer to short-chained oligosaccharides, also including disaccharides. The enzymatic hydrolysis is performed by methods known per se using cellulose-disintegrating enzymes, i.e. cellulases.

The sugar product of the invention may be a glucose product, for example.

The sugar product thus obtained is useful as a raw material for the manufacture of different industrial chemicals.

In an embodiment of the invention, the sugar product thus obtained is subjected to fermentation into ethanol by methods known per se. The fermentation may be performed for instance as follows: the sugar product is fed as an aqueous solution into a fermentor, wherein the yeast Saccharomyces cerevisiae converts the soluble sugars into ethanol and carbon dioxide. The residence time in the fermentor is typically 48 hours and the temperature 32° C.

In connection with the above-mentioned separations, the carbohydrate fraction may be in a suspension together with a mixture of organic acids. The total acid concentration of this mixture may vary between 0 and 98% without the suspension containing practically any dissolved organic matter. Such a suspension may be brought to react, whereby the polysaccharides of the carbohydrate fraction are converted into a more easily hydrolyzable form, but the polysaccharides do not, however, react much into oligosaccharides or monosaccharides or dissolve. At the same time, the organic acids that were attached chemically to the solid carbohydrate fraction as esters are released from the carbohydrate fraction. The release of acids bound as esters significantly reduces the acid losses of the method. Because the suspension only contains a small amount of dissolved organic matter, practically no disintegration products are generated in the treatment, such as furfural or hydroxymethylfurfural, which could complicate the enzymatic hydrolysis and the fermentation.

Thus, the treatment of the carbohydrate fraction obtained in step (A) according to the method of the invention may be continued, further facilitating the treatment of the solid carbohydrate fraction, and some polysaccharides react into water-soluble oligosaccharides and monosaccharides.

In an additional embodiment of the invention, the method may thus include also one or more steps, wherein the solid carbohydrate fraction is treated further with a reagent containing one or more organic acids, whereby a further-treated carbohydrate fraction, a fraction/fractions containing used organic acids and possibly a fraction containing dissolved organic matter are obtained. The reagent used in the further treatment may be the same as the one used in the first fractionation. In the further treatment, the reaction mixture to be treated is typically heated to a range of 60 to 220° C. The treatment time may be within a range of 1 minute to 72 h. The further-treated carbohydrate fraction thus obtained is then separated from the fraction containing used organic acids.

The further treatment may be performed for instance under the following conditions: treatment reagent a formic acid mixture containing 1 to 50% formic acid, temperature 100 to 180° C., residence time 10 minutes to 24 h, solid matter content of reaction mixture 2 to 40%.

The further treatment is performed after step (A), before the fractions obtained in the fractionation are separated from each other or in connection with the separation of the fractions or thereafter. In an embodiment, the further treatment is performed in connection with a two-step separation of the fractions by adding concentrated washing acid in the first washing step to the carbohydrate fraction obtained, yielding a suspension composed of the carbohydrate fraction and the washing acid, the temperature of the suspension is raised to a temperature of 60 to 220° C., the suspension is allowed to react at this temperature e.g. for 10 minutes to 24 h, followed by washing the further-treated carbohydrate fraction with water in the second washing step. The concentrated washing acid may be the same acid mixture as was used in the first fractionation.

In an embodiment of the invention, said further treatment is performed under conditions wherein more than 90% of the solid carbohydrate fraction remains in a solid form. Such conditions may be for instance the following: treatment reagent a formic acid mixture containing 1 to 50% formic acid, temperature 100 to 160° C., residence time 10 minutes to 4 h, solid matter content of reaction mixture 2 to 40%.

In another embodiment of the invention, the further treatment is performed under conditions wherein more than 10% of the polysaccharides of the solid carbohydrate fraction react into water-soluble monosaccharides and oligosaccharides, whereby a further-treated solid carbohydrate fraction and a fraction/fractions containing water-soluble monosaccharides and oligosaccharides and used organic acids are obtained. Such conditions may be for instance the following: treatment reagent a formic acid mixture containing 1 to 50% formic acid, temperature 130 to 180° C., residence time 1 to 8 h, solid matter content of reaction mixture 2 to 40%.

The fractions obtained from the further treatment are then separated from each other by the above-mentioned known methods, such as filtering, washing or pressing.

The fraction obtained and containing water-soluble monosaccharides and oligosaccharides and organic acids may be further fractionated into a fraction containing water-soluble monosaccharides and oligosaccharides and a fraction containing used organic acids. Since organic acids are easily vaporized, this separation may be suitably performed by thermal separation operations, such as evaporation, for example.

The further-treated solid carbohydrate fraction is then enzymatically hydrolyzed in step (B) into water-soluble monosaccharides and oligosaccharides in the same manner as above. The concentrated saccharide fraction obtained from the further treatment and containing water-soluble monosaccharides and oligosaccharides does not necessarily require enzymatic hydrolysis, but is usable as such for upgrading. Alternatively, the concentrated saccharide fraction may be hydrolyzed further into monosaccharides. The hydrolyzed monosaccharides and oligosaccharides may be upgraded into ethanol, for example.

The oligosaccharide and monosaccharide products obtained from the further treatment are also useful as raw material for the manufacture of different industrial chemicals in the same manner as above. In an embodiment of the invention, the oligosaccharides and the monosaccharides are hydrolyzed into ethanol.

The used organic acids and washing filtrates are recovered and purified. In the method, acetic acid and furfural may also be formed, which are separated and are useful as industrial products.

Between steps (A) and (B), the method of the invention may also include a step wherein the fibrous and non-fibrous materials in the carbohydrate fraction obtained in step (A) are separated from each other, yielding a fraction containing fibrous material and a fraction containing non-fibrous material. In an embodiment of the invention, the enzymatic hydrolysis of step (B) is performed only on one of these fractions.

Before step (A), the method of the invention may also include a step wherein the organic acids used as the washing reagent are absorbed into the biomass being treated.

When the carbohydrate fraction is treated with a mixture of organic acids, the hemicelluloses are more easily hydrolyzed than the cellulose. By the method of the invention, the hemicelluloses may be hydrolyzed without the cellulose being practically at all hydrolyzed. Hydrolyzed hemicelluloses dissolve as oligosaccharides and monosaccharides that can be recovered from washing filtrates and used as raw material for the manufacture of furfural, for example.

In a practical embodiment, the method of the invention typically includes the following steps: treatment of biomass with a mixture of organic acids, separation of dissolved material from the solid carbohydrate fraction obtained, separation of cooking acids from the solid carbohydrate fraction by washing with water, enzymatic hydrolysis of the carbohydrate fraction, fermentation of the glucose and the oligosaccharides obtained as hydrolysis products and separation of the fermentation products (ethanol), recovery of the washing acids, purification of the washing acids and the water, recovery of the chemicals generated in the process, such as acetic acid and furfural, and recovery of the dissolved organic matter.

In a practical embodiment, the separation of cooking acids from the solid carbohydrate fraction may comprise two washing steps, between which the mixture containing the carbohydrate fraction is heated. In another practical embodiment, part of the cooking acid is first separated by washing, followed by heating the mixture containing the carbohydrate fraction under conditions wherein part of the saccharides is dissolved, followed by separation of the remaining acid and the dissolved saccharides by washing, and, finally, separating the dissolved saccharides by evaporation.

In a third practical embodiment associated with the separation of cooking acids, part of the acid is first separated by washing, followed by heating the mixture containing the carbohydrate fraction, separating the cooking acids and dissolved pentosans by washing, separating the dissolved saccharides by evaporation, heating the solid carbohydrate fraction thus obtained under conditions wherein part of the saccharides is dissolved, and finally separating the remainder of the cooking acid and the dissolved saccharides by washing.

In the following, the invention will be described by illustrative, but not restrictive examples. In the examples and the entire description and the claims, the percentage contents are percentages by weight (w-%), unless otherwise stated.

### Example 1

Three fractionations A, B and C were performed with organic acids by using wheat straw as the starting material.

The content of pentosans and the content of lignin (kappa number) were measured from the solid carbohydrate fractions obtained from the fractionations. The enzymatic hydrolyzability of the different fractions was compared with a cellulase dose of 60 FPU (‘Filter Paper Unit’). The cellulase enzyme used was commercial cellulase GC 200 (manufacturer Genencor). The yield of the hydrolysis product, i.e. glucose, was calculated as follows: (1) the cellulose content of the sample was estimated based on the kappa number, the pentosan content and the ash content, (2) the amount of glucose obtained from the enzymatic hydrolysis was divided by the estimated cellulose content, and (3) the ratio obtained was multiplied by the ratio of the cellulose unit to the molar masses of the glucose (162/180).

The fractionation conditions and the results are presented in the following table.

| Fractionation test | A | B | C |
|---|---|---|---|
| Composition of liquid to be fed, | | | |
| w-% | | | |
| c(formic acid) | 42 | 42 | 85 |
| c(acetic acid) | 40 | 36 | 0 |
| c(water) | 18 | 22 | 15 |
| Temperature, ° C. | 141 | 150 | 148 |
| Reaction time, min | 27 | 30 | 40 |
| Composition of solid fraction and | | | |
| properties after fractionation | | | |
| Kappa number | 11 | 15 | 32 |
| Pentosans, w-% | 6 | 4 | 2 |
| Ash, w-% | 12 | 12 | 12 |
| Enzymatic hydrolysis | | | |
| of solid fraction | | | |
| Production of glucose (12 h), | 61 | 67 | |
| % of solid fraction | | | |
| Production of glucose (72 h), | 69 | 74 | 78 |
| % of solid fraction | | | |
| Yield of hydrolysis (12 h), % | 69 | 73 | |
| Yield of hydrolysis (72 h), % | 77 | 82 | 86 |

From the results, the conclusion can be drawn that, the hydrolyzability of the carbohydrate fraction obtained can be affected by the fractionation conditions. It was also found that carbohydrate fractions B and C are poorly suitable for use as pulp, since the pentosan content thereof is lower and the lignin content higher than those of fraction A. The higher lignin content is partly the result of the condensation of the dissolved lignin back into the solid fraction.

Carbohydrate fractions B and C contain only a small amount of pentosans and lignin relative to the original biomass, and they are therefore well suitable for enzymatic hydrolysis and for further fermentation into ethanol.

### Example 2

The carbohydrate fraction B of Example 1 was further treated with an acid mixture containing 10 w-% formic acid and 90 w-% water, at a temperature of 130° C., 90 min. At the start of the treatment, the suspension contained 7.5% solid matter.

The contents of bound acids before and after the treatment were measured. The enzymatic hydrolyzability of the carbohydrate fractions was compared (enzyme dose 60 FPU).

The results are presented in the following table.

| | Carbohydrate fraction | |
|---|---|---|
| | from fractionation | Carbohydrate |
| | experiment B before | fraction after |
| | further processing | further processing |
| Bound acids in carbohydrate | | |
| fraction, w-% | | |
| formic acid | 1.8 | 0.2 |
| acetic acid | 2.1 | 0.1 |
| Pentosans, w-% | 4 | 3 |
| Enzymatic hydrolysis | | |
| of solid fraction | | |
| Production of glucose (12 h), | 67 | 84 |
| % of solid fraction | | |
| Production of glucose (72 h), | 74 | 88 |
| % of solid fraction | | |
| Yield of hydrolysis (12 h), % | 73 | 92 |
| Yield of hydrolysis (72 h), % | 82 | 95 |

The conclusion can be drawn that the hydrolyzability can be improved and the bound acids released by further treatment without practically hydrolyzing the carbohydrate fraction at all.

### Example 3

The carbohydrate fraction B of Example 1 was further treated with an acid mixture containing 30 w-% formic acid and 70 w-% water, at a temperature of 160° C., 90 min. At the start of the treatment, the suspension contained 7.5% solid matter.

The enzymatic hydrolyzability of the carbohydrate fractions was compared (enzyme dose 60 FPU). The contents of the liquid part of the suspension were measured for glucose and hydroxymethylfurfural, which is the main disintegration product of glucose under acidic conditions.

| | Carbohydrate fraction | |
|---|---|---|
| | from fractionation | Carbohydrate |
| | experiment B before | fraction after |
| | further processing | further processing |
| Composition of liquid | | |
| part of suspension, w-% | | |
| of original dry matter | | |
| Glucose | — | 5 |
| Hydroxymethylfurfural | — | 0.2 |
| Pentosans, w-% | 4 | 3 |
| Enzymatic hydrolysis | | |
| of solid fraction | | |
| Production of glucose (12 h), | 67 | 90 |
| % of solid fraction | | |
| Production of glucose (72 h), | 74 | 93 |
| % of solid fraction | | |
| Yield of hydrolysis (12 h), % | 73 | 96 |
| Yield of hydrolysis (72 h), % | 82 | 99 |

In the further treatment, 23% of the solid carbohydrate fraction reacted into a soluble form.

From the experiment, the conclusion may be drawn that the hydrolyzability can be further improved by further treatment without practically any loss of glucose due to disintegration reactions. The material dissolved is mainly glucose and oligosaccharides formed by glucose units (e.g. cellobiose).

### Example 4

The carbohydrate fraction C of Example 1 was further treated with an acid mixture containing 30 w-% formic acid and 70 w-% water, at a temperature of 130° C., 180 min. At the start of the treatment, the suspension contained 7.5% solid matter.

The enzymatic hydrolyzability of the carbohydrate fractions was compared (enzyme dose 15 FPU).

The results are presented in the following table.

| | Carbohydrate fraction | |
|---|---|---|
| | from fractionation | Carbohydrate |
| | experiment C before | fraction after |
| | further processing | further processing |
| Pentosans, w-% | 2 | 1.5 |
| Enzymatic hydrolysis | | |
| of solid fraction | | |
| Production of glucose (12 h), | | 87 |
| % of solid fraction | | |
| Production of glucose (72 h), | 78 | 89 |
| % of solid fraction | | |
| Yield of hydrolysis (12 h), % | | 95 |
| Yield of hydrolysis (72 h), % | 86 | 98 |

The conclusion can be drawn that the hydrolyzability can be improved by further treatment, and that enzymatic hydrolysis operates fast also at a low enzyme dosage, i.e. after the treatment, the carbohydrates are in an easily hydrolyzable form.

### Example 5

The carbohydrate fraction B of Example 1 was further treated with an acid mixture containing 30 w-% formic acid and 70 w-% water, at a temperature of 160° C., 30 min. At the start of the treatment, the suspension contained 7.5% solid matter. After the treatment, the liquid part of the suspension contained xylose 1.7 g/l and glucose 0.6 g/l. Consequently, the xylans contained by the carbohydrate fraction can be hydrolyzed into xylose practically without hydrolyzing the glucose part of the fraction at all.

### Example 6

Fine matter was separated from unbleached pulp made from Miscanthus sinensis and treated in an acid mixture under the following conditions: 80 w-% formic acid and 20 w-% water, temperature 160° C., reaction time 240 min. Thus, although the conditions were clearly harder than in Example 3, only 7% of the solid carbohydrate fraction reacted into a soluble form. Accordingly, the hydrolysis of the fine matter was significantly more difficult than the hydrolysis of the carbohydrate fraction of Example 3, i.e. the different solid carbohydrate fractions react in very different manners in acid treatment.

### REFERENCES

1. Hamelinck, C. N, van Hooijdonk, G. & Faaij, A. P. C, Ethanol from lignocellulosic biomass: techno-economic performance in short-, middle- and long-term, Biomass and Bioenergy 28: 384-410, 2005.
2. Yang, B. & Wyman, C. E., Effect of Xylan and Lignin Removal by Batch and Flowthrough Pretreatment on the Enzymatic Digestibility of Corn Stover Cellulose, Biotechnology and Bioengineering 86 (1): 88-95, 2004.
3. U.S. Pat. No. 5,726,046 (published 10 Mar. 1998), Farone, W. & Cuzens, J., Method of producing sugars using strong acid hydrolysis.
4. U.S. Pat. No. 4,904,342 (published 27 Feb. 1990), Arnoldy, P. & Petrus, L., Process for pulping lignocellulose containing material.
5. Bucholtz, M. & Jordan, R. K., Formic acid woodpulping could yield valuable chemical products, Pulp & Paper 57 (9): 102-104, 1983.
6. DE patent 3 445 132 (published 12 Jun. 1986), Nimz, H. H. & Casten, R., Holzaufschluss mit Essigsäure.
7. WO 03/006737 A1 (published 23 Jan. 2003), Rousu E., Rousu P., Anttila J. & Rousu, P., Process for producing pulp.
8. WO 98/20198 (published 14 May 1998), Rousu, P., Rousu, P. & Rousu, E., Method for producing pulp using single-stage cooking with formic acid and washing with performic acid.
9. WO 86/05529 (published 25 Sep. 1986), Laamanen, A. K., Sundquist, J. J., Wartiovaara, I. Y. P., Kauliomaki, S. V. M., Poppius, K. J., Process for preparing bleached pulp out of lignocellulosic raw material.
10. U.S. Pat. No. 5,681,427 (published 28 Oct. 1997), Lora J. H., Maley, P., Greenwood, F., Phillips, J. R., Lebel, D. J., Apparatus for treating pulp produced by solvent pulping.
11. Pan, X., Gilkes, N., Kadl, J., Pye, K., Saka, S., Gregg, D., Ehara, K., Xie, D., Lam, D. & Saddler, J., Bioconversion of hybrid poplar to ethanol and co-products using an organosolv fractionation process: Optimization of process yields, Biotechnology and Bioengineering 94 (5): 851-861, 2006.
12. Rousu P, Rousu P & Pohjola V J (2000). Nonwood fibres made by formic acid based pulping method—holistic characterization of paper product. Proc. Fourth International Nonwood Fibre Pulping and Papermaking Conference, Sep. 18-21, 2000, Jinan, P. R. China, CTAPI 2000, 1: p. 140-151.
13. WO 02/053829 A1 (published 11 Jul. 2002), Rousu, E., Rousu P., Anttila, J., Tanskanen, J. & Rousu, P., Method for producing furfural, acetic acid and formic acid from spent pulp-cooking liquor.
14. Rousu, P. P, Rousu, P., Anttila, J. R., & Tanskanen, J. P., A novel biorefinery—production of pulp, bioenergy and green chemicals from nonwood materials. Proceedings of TAPPI Engineering, Pulping & Environmental Conference, 2006.

1. A method for producing sugars from lignocellulose-containing biomass, characterized by
(A) treating the biomass with a reagent containing one or more organic acids, yielding a solid carbohydrate fraction having improved hydrolyzability, and a fraction/fractions containing organic matter dissolved from the biomass and used organic acids, and
(B) enzymatically hydrolyzing at least part of the solid carbohydrate fraction obtained into water-soluble monosaccharides and oligosaccharides, yielding a sugar product.

2. A method as claimed in claim 1, characterized in that the treatment reagent of step (A) contains formic acid and/or acetic acid.

3. A method as claimed in claim 1, characterized in that the treatment temperature in step (A) is 60 to 220° C.

4. A method as claimed in claim 1, characterized in that the solid carbohydrate fraction obtained from step (A) contains mainly polysaccharides composed of glucose units, and, in addition less than 10% polysaccharides composed of pentose units.

5. A method as claimed in claim 1, characterized in that the solid carbohydrate fraction obtained from step (A) contains fibrous and non-fibrous material.

6. A method as claimed in claim 1, characterized in that the treatment reagent of step (A) contains less than 60% acetic acid, the remainder being formic acid and water.

7. A method as claimed in claim 6, characterized in that the amount of formic acid is at least 20%.

8. A method as claimed in claim 1, characterized in that the treatment temperature of step (A) is 100 to 180° C.

9. A method as claimed in claim 1, characterized in that the method further includes, between steps (A) and (B), one or more steps wherein the solid carbohydrate fraction of step (A) is further treated with a reagent containing one or more organic acids, yielding a further-treated solid carbohydrate fraction, a fraction/fractions containing used organic acids, and, optionally, a fraction containing dissolved organic matter, after which the further-treated carbohydrate fraction obtained is subjected to the enzymatic hydrolysis according to step (B), yielding a sugar product.

10. A method as claimed in claim 9, characterized by performing the further treatment under conditions wherein more than 90% of the solid carbohydrate fraction remains in solid form.

11. A method as claimed in claim 9, characterized by performing the further treatment under conditions wherein more than 10% of the polysaccharides of the solid carbohydrate fraction react into water-soluble monosaccharides and oligosaccharides, yielding a further-treated solid carbohydrate fraction and a fraction/fractions containing water-soluble oligosaccharides and polysaccharides and used organic acids.

12. A method as claimed in claim 11, characterized by fractionating the fraction/fractions containing water-soluble monosaccharides and oligosaccharides and used organic acids, yielding, as a sugar product, a fraction containing water-soluble monosaccharides and oligosaccharides, and a fraction containing used organic acids.

13. A method as claimed in claim 9, characterized in that the treatment temperature is 60 to 220° C.

14. A method as claimed in claim 11, characterized by enzymatically hydrolyzing the solid carbohydrate fraction and/or the fraction containing water-soluble monosaccharides and oligosaccharides into water-soluble monosaccharides and oligosaccharides or into monosaccharides, yielding a sugar product.

15. A method as claimed in claim 1, characterized by fermenting the sugar product obtained into ethanol.

16. A method as claimed in claim 1, characterized by recovering the used organic acids.

17. A method as claimed in claim 1, characterized by recovering the dissolved organic matter.

18. A method as claimed in claim 1, characterized in that the method further includes, between steps (A) and (B), a step for separating the fibrous and non-fibrous materials from each other, yielding a fraction containing fibrous material and a fraction containing non-fibrous material.

19. A method as claimed in claim 18, characterized by performing the enzymatic hydrolysis according to step (B) only on one of the fractions separated.

20. A method as claimed in claim 1, characterized in that the method further includes, before step (A), a step of absorbing the organic acids used as cooking reagents into the biomass.
